(19) United States
(12) Patent Application Publication
(10) Pub. No.: US 2009/0005786 A1
(43) Pub. Date: Jan. 1, 2009

(54) Bone hole measuring device

(75) Inventors: Ole Prien, Kiel (DE); Helge Giersch, Kiel (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Trauma GmbH, Schonkirchen (DE)

(21) Appl. No.: 11/824,590

(22) Filed: Jun. 28, 2007

(51) Int. Cl. A61B 17/58

(52) U.S. Cl. 606/102

(57) ABSTRACT

A measuring device for measuring the length of a through-hole in a bone has an elongated hollow sleeve preferably having an outer cross-section adapted to a cross-section of the through-hole and having an opening at its distal extremity. The measuring device further comprises a rod having a hook-shaped end at its distal extremity. The rod is at least partly housed within the hollow sleeve and is slidable with respect to the sleeve along its longitudinal axis. The sleeve and the rod are designed such that, when the rod is slid to a distal position of the sleeve, the hook-end can be displaced such as to protrude through the opening of the sleeve and laterally over the lateral outer surface of the sleeve. When the rod is slid to a proximal position of the sleeve, the rod no longer protrudes laterally over the outer surface of the sleeve.

### BACKGROUND OF THE INVENTION

The present invention relates to a measuring device and to a measuring kit for measuring a length of a through-hole in a bone, such as a long bone, for example, a femur, tibia and humerus.

It is known, for example, in osteosynthesis, to provide a connection between an implant and a bone by elongated fixing elements such as screws. The fixing element may be inserted into the bone bicortically i.e. the fixing element extends into or through both cortex-parts at opposing surfaces of the bone. A through-hole through the bone having a diameter corresponding to the core diameter of the screw to be used for fixing the implant to the bone is usually drilled before inserting the screw. For this purpose, a hollow protection sleeve may be inserted through the patient's soft tissue surrounding the bone until it abuts the bone. Then, a drill may be inserted through the protection sleeve and may drill the through-hole into the bone.

After pre-drilling the through-hole, a surgeon may have to select the screw having the correct length such that the screw extends into both of the opposing cortex regions of the bone while at the same time not protruding over the bone surface at the side of the bone opposite to the implant to be fixed on the bone.

In order to measure the thickness of the bone at the location of the pre-drilled through-hole, i.e. the length of the through-hole, a surgeon conventionally uses a measuring hook being simply a bar having a bent end section having an L-shape. The bar has a diameter substantially smaller than the diameter of the through-hole. For measuring, the hook is inserted through the protection sleeve and through the through-hole and then the surgeon tries to hook the L-shaped hook-end into the opposite distal cortex of the bone. From the relative position of the hook-end attached to the distal cortex and the protection sleeve abutting the proximal cortex, the surgeon can determine the length of the through-hole and accordingly the correct length of the screw to be used.

However, when using conventional measuring hooks, it may be difficult to fix the hook at the distal cortex as the hook may slip off the rim of the through-hole. Furthermore, there may be a risk of accidentally clamping some soft tissue between the distal hook-end and the cortex which, on the one hand, may falsify the measurement and, on the other hand, may injure the soft tissue.

There is a need for a measuring device or a measuring kit for measuring a length of a through-hole in a bone wherein the measuring device or the measuring kit can be fixed easily and reliably with respect to a through-hole in a bone or with respect to surface around such through-hole and where the risk of an incorrect measurement or injuring soft tissue is reduced.

### SUMMARY OF THE INVENTION

As used herein, the terms “distal” and “proximal” can be interpreted such that distal means away from a surgeon or from a position from which access to the through-hole can be obtained during an operation whereas proximal means closer to a surgeon.

According to a first aspect of the present invention, a measuring device for measuring a length of a through-hole in a bone is proposed. The measuring device comprising an elongated hollow sleeve having an opening at its distal extremity adjacent the second cortical bone wall. Preferably, an outer cross-section of the sleeve is adapted to a cross-section of the through-hole. The measuring device further comprises a bar or rod having a hook-end at its distal extremity. The bar or rod is at least partly housed within the hollow sleeve and is slidable with respect to the sleeve along its longitudinal direction. Furthermore, the sleeve and the bar are adapted such that, slid to a distal position of the sleeve, the hook can be displaced such as to protrude through the opening of the sleeve and laterally over the lateral outer surface of the sleeve, and such that, when slid to a proximal position of the sleeve, the bar does not protrude laterally over the outer cross-section of the sleeve.

According to the present invention the measuring device is provided having a hollow sleeve and a rod slidably guided therein. The sleeve can be inserted into the through-hole the length of which has to be measured. As the outer cross-section of the sleeve is preferably adapted such as to substantially match the inner cross-section of the through-hole, the sleeve may be fixedly held within the through-hole while measuring the length of the through-hole. While inserting the sleeve into the through-hole, the rod may be located in the proximal position such as to not protrude laterally over the cross-section of the sleeve. After insertion of the sleeve into the through-hole, the rod can be brought to the distal position in which the hook-end protrudes laterally over the lateral surface of the sleeve. The measuring device can then be drawn back in the through-hole until the laterally protruding hook comes into abutment with the laterally surrounding surface of the bone, i.e. its cortex. In this position, the measuring device may be securely and fixedly held within the through-hole and there is no risk of the hook slipping off the cortex as the sleeve is securely held within the through-hole and the hook-end laterally protrudes over the surface of the sleeve.

In the following, possible details and advantages of the present invention according to the first aspect and embodiments thereof will be explained.

Through-holes prepared in a bone are usually created by drilling such that they have a circular cross-section. Accordingly, the hollow sleeve may have a circular cross-section and may be, for example, in the form of a cylindrical tube. The outer cross-section or diameter of the sleeve may be selected such as to substantially correspond to the inner cross-section or diameter of the through-hole. In that way, the sleeve may be press-fit into the through-hole while measuring. Typical diameters for through-holes in bones are in the order of a few millimetres.

At its distal extremity and preferably at or close to its distal end, the sleeve has an opening. For example, the opening can extend in a lateral surface of the sleeve and can be provided as a longitudinal slit in the sleeve.

The bar or rod to be fixed on a surface adjacent to the through-hole for measuring purposes and therefore having a hook-end at its distal extremity is usually, i.e. in the “proximal position” of the bar, completely contained within the hollow sleeve and does not protrude there from. The bar may have an outer cross-section corresponding to the inner cross-section of the hollow sleeve such that there is substantially no or little lateral play between the bar and the sleeve. However, the bar should be slidable along the longitudinal direction of the sleeve. By longitudinally sliding the bar into a specific position, named herein as “distal position” of the sleeve, the bar or its position can be modified such that its hook-end protrudes through the opening of the sleeve and laterally over the surface of the sleeve. By doing so, the local outer cross-section of the entire measuring device is increased at the position where the hook-end protrudes laterally.

For measuring the length of the through-hole, the distal end of the measuring device is slid through the through-hole with the bar in the non-laterally protruding proximal position until the location of potentially increased cross-section is located distally (behind) of the through-hole. The bar will then be brought into the protruding distal position such that the cross-section is increased locally by the protruding hook-end. The measuring device can then be drawn into the proximal direction until the location of increased cross-section abuts the cortex surrounding the through-hole. The length or depth of the through-hole may then be read from a scale formed on the outer surface of the sleeve.

According to an embodiment of the present invention the sleeve is substantially rigid and at least the distal extremity of the bar is substantially flexible such as to be deflectable into a bent configuration to protrude over the lateral surface of the sleeve. In other words, the sleeve is adapted not to be deflected while measuring the through-hole in the bone and accordingly the sleeve can act as a rigid guide to the bar or rod housed at least partly therein. The bar, at least at its distal extremity, has a certain degree of flexibility. Due to this flexibility, the bar can be deflected by suitable means provided for example at the sleeve end (to be described further below) such as to protrude over the lateral surface of the sleeve thereby increasing the local cross-section or diameter of the measuring device. When brought to the proximal position the bar may resume its original undeflected shape where its hook-end does not laterally protrude. For example, the sleeve may be a rigid metal tube and the bar or rod may be a flexible metal wire housed within this tube.

According to a further embodiment, the hollow sleeve comprises an angled or titled ramp at its inner side such that the ramp may force the distal extremity of the rod into the deflected configuration when the bar is slid to the distal position of the sleeve. In other words, at its bore inner side, the hollow sleeve may have a region which is adapted to deviate the preferably flexible distal extremity of the bar when the bar is pushed distally within the sleeve. Accordingly, in its proximal position, the bar may be located away from the ramp and be housed completely within the hollow sleeve. In this position, the measuring device can be easily inserted into the through-hole to be measured. Then the rod can be pushed linearly to a distal position within the sleeve. Due to the ramp, the distal extremity of the rod or bar having the hook-end thereon will be deviated from its linear direction and will be pushed laterally out of the opening at the distal extremity of the hollow sleeve. Thereby, the hook-end will protrude over the outer cross-section of the sleeve.

According to a further embodiment the sleeve extends longitudinally beyond the position of the hook-end being in its distal protruding position. In other words, the end of the sleeve is at a further distal position than the position of the hook-end when the bar is slid to the most distal position. Accordingly, when the measuring device is inserted into a patient's body, the distal end of the sleeve first pushes away any soft tissue which otherwise might interfere with the measurement. For example, when the measuring device is inserted through the through-hole to be measured, it will exit from the distal opening of the through-hole. As the sleeve extends distally beyond the hook-end, the sleeve will push away any soft tissue surrounding the through-hole such that there is no risk of clamping soft tissue when the laterally protruding hook-end is drawn into abutment with the distal cortex of the bone.

According to a further embodiment the distal extremity of the hollow sleeve is tapered. In other words, the outer cross-section of the sleeve reduces towards the distal end of the sleeve. For example, the distal end of the sleeve may be rounded or may have a tip in a cone-shape. The tapered distal extremity of the sleeve can be easily inserted into the through-hole in the bone the thickness of which is to be measured.

According to a further embodiment, the measuring device comprises a counter-piece or indicator being slidable with respect to the sleeve and being adapted for indication of a distance between the hook-end of the bar and a distal end of the counter-piece. In other words, additionally to the sleeve housing the rod, there is a counter-piece which may be slid along the longitudinal direction of the sleeve. Accordingly, for measuring the thickness of a bone at the location of a through-hole, the sleeve can be inserted into the through-hole and the hook-end of the rod can protrude laterally from the sleeve and brought into abutment to the distal surface of the bone. Subsequently, the counter-piece can be slid along the sleeve in order to come into abutment with the proximal surface of the bone. The measuring device may then comprise a scale which indicates the distance between the hook-end of the bar and the distal end of the counter-piece thereby indicating the thickness of the bone positioned therebetween.

For example, both the sleeve and the counter-piece can be provided as cylindrical tubes, the sleeve having a slightly smaller diameter than the counter-piece such that the inner surface of the counter-piece can easily slide along the outer surface of the sleeve. In such embodiment, the counter-piece may also be used as hollow protection sleeve through which a drill may be guided when pre-drilling the through-hole. After drilling the through-hole, the counter-piece may remain in its position abuting the proximal cortex of the bone and the drill may be replaced by the sleeve-bar-combination of the measuring device for measuring the length of the through-hole.

According to a further embodiment, the measuring device further comprises a handle which can be attached to a proximal extremity of the sleeve and of the rod. Therein, the handle is adapted to displace the bar or rod longitudinally along the sleeve upon activation of the handle. In other words, a handle can be provided which may be adapted to push and/or draw the rod linearly within the sleeve when the handle is activated in a specific way. When the handle is activated in order to push the rod to the distal position, this activation also induces protruding of the hook-end of the bar over the lateral surface of the sleeve. On the other side, when the handle is activated to draw the rod to the proximal position, the measuring device is brought into a state where the hook-end does not protrude such that the sleeve can be inserted into the through-hole.

Different kinds of possibilities of activation of the handle can be realized. According to a further embodiment, the handle is adapted to be activated by rotating a part of the handle. For example, the handle may comprise two parts, namely a static grip and a rotatable part which can be rotated around the longitudinal axis of the sleeve. Such two-piece handle allows an easy handling of the measuring device. For example, the sleeve can be inserted into the through-hole to be measured and then the bar or rod can be made to laterally protrude simply by turning the rotatable part of the handle.

Additionally, the handle can also be adapted such that the counter-piece can be attached to it. For example, the handle can be fixed to the counter-piece such that by pushing or withdrawing the handle, the counter-piece can be slid along the sleeve and can be brought into abutment with the proximal surface of the bone. In contrast to this linear movement of the handle for displacing the counter-piece, the handle can be rotated for activating the bar in order to bring it to the distal protruding position.

According to a further embodiment, the handle comprises an adapter such that the sleeve, the bar and/or the counter-piece can be releasably attached to the handle. For example the sleeve can be attached to the handle using a threading, a snap-in lock or a quick-release lock. Using such releasable attachment, the sleeve, the bar and/or the counter-piece can be easily removed from the handle e.g. for sterilization purposes. Furthermore, units including a sleeve and a matching bar and/or counter-pieces of different outer cross-sections can be attached to and released from the handle. Accordingly, the measuring device can be used for measuring through-holes of different diameters simply by replacing the sleeve by a sleeve of a suitable diameter.

According to a further aspect of the present invention, a bone thickness measuring kit is provided comprising a measuring device as described further above and further comprising a plurality of replacement sleeves having different outer cross-sections. Accordingly, for measuring a specific through-hole, the measuring device can be equipped with a replacement sleeve having a cross-section substantially corresponding to the cross-section of the through-hole.

The parts of the measuring kit and especially the replacement sleeves may be made from a material such as a metal like stainless steel which can be easily sterilized after each measuring process or may be provided as single-use pieces made for example from pre-sterilized inexpensive plastics.

It has to be noted that embodiments of the invention are described with reference to different subject-matters. In particular, some embodiments are described with reference to the measuring device whereas other features are described with reference to a method of using same. However, a person skilled in the art will gather from the above and the following description that, unless otherwise notified, in addition to any combination of features belonging to one type of subject-matter also any combination between features relating to the different subject-matters, in particular between features of the apparatus type claims and features of the method, are considered to be disclosed with this application.

The aspects defined above and further aspects, features and advantages of the present invention can be derived from the examples of embodiments described hereinafter.

The invention will be described in more detail hereinafter with reference to examples of embodiments but to which the invention is not limited.

### BRIEF DESCRIPTION OF THE DRAWINGS

FIG. 1 shows a perspective schematic overview of a measuring device according to an embodiment of the present invention;

FIG. 2 shows a perspective view onto the distal extremity of a measuring device according to an embodiment of the present invention;

FIG. 3 shows a side view of the distal extremity of the measuring device shown in FIG. 2;

FIG. 4 shows a longitudinal cross-section of the measuring device shown in FIG. 2;

FIG. 5 shows an perspective explosion view from a first view point of a measuring device according to an embodiment of the present invention;

FIG. 6 shows an perspective explosion view from a different view point of the embodiment shown in FIG. 5;

FIG. 7 shows an enlarged side view of the region A indicated in FIG. 5; and

FIG. 8 shows a partial cross-section of the assembled measuring device of FIGS. 5 and 6.

In the above figures, like reference signs designate like elements. Furthermore, it is to be noted that the figures are only schematically and not to scale.

### DETAILED DESCRIPTION

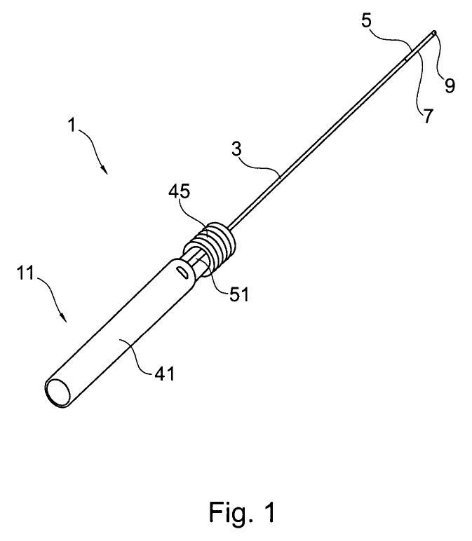

FIG. 1 schematically shows a preferred measuring device 1 for measuring the length of a through-hole in a bone for example a long bone such as a tibia or femur, including an elongated hollow sleeve 3 with an opening 5 at its distal extremity and a bar or rod 7 having a hook-end 9 at its distal extremity. The measuring device 1 further comprises a handle 11 including a rotatable grip 41, a static part 45 and a scale 51, wherein the rotatable grip 41 is adapted for displacing the bar or rod 7 longitudinally along sleeve 3.

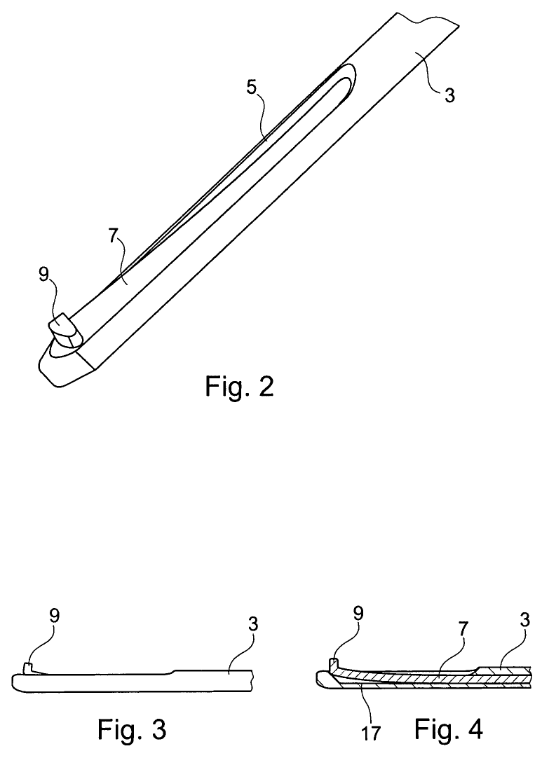

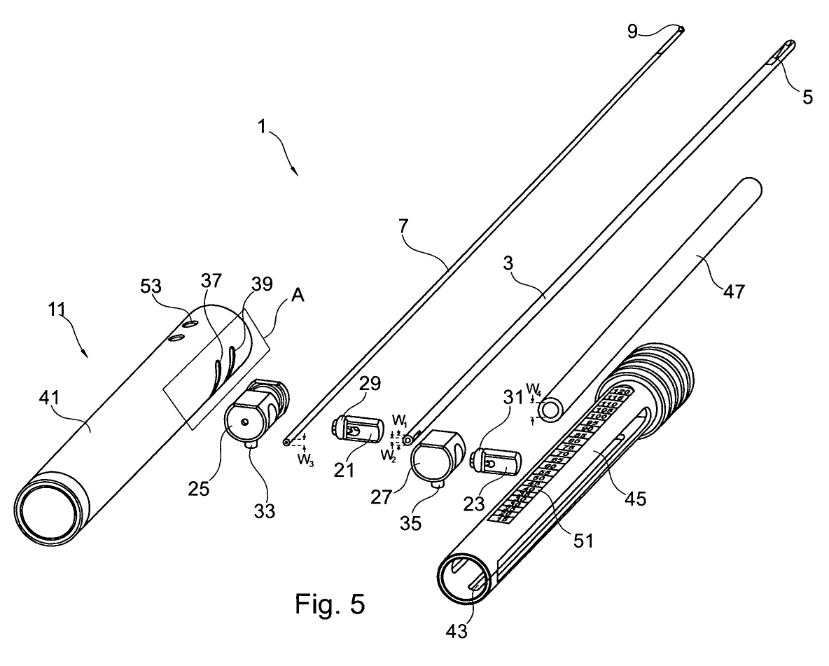

As can be seen from FIGS. 2, 3 and 4, the distal extremity of preferred sleeve 3 comprises an opening 5 in the form of an elongated slot in the lateral surface of sleeve 3. In the preferred embodiment the opening 5 has a width corresponding to the width of rod 7 housed within sleeve 3 such that rod 7 can deflect out of sleeve 3 through opening 5 at the distal part of sleeve 3.

In a distal position where rod 7 is pushed towards the distal extremity of sleeve 3, the distal end of rod 7 having the hook-end 9 formed thereon is pushed along a surface of a ramp 17 formed at the inner side at the most distal extremity of sleeve 3. Thereby, the distal extremity of rod 7 having substantial mechanical flexibility, is forced laterally out of opening 5 (upwards in the drawings) such that the hook-end 9 protrudes over the lateral outer surface of sleeve 3.

Brought to a proximal position (not shown in the figures), rod 7, including hook-end 9, can be completely housed within the bore or hollow interior of such that the hook-end 9 does not protrude over the lateral surface of sleeve 3.

In the preferred embodiment the distal end of sleeve 3 is tapered and rounded in order to be able to easily insert sleeve 3 into a pre-drilled through-hole within a bone without risking to damage the cortex of the bone.

As shown in FIGS. 5, 6 and 8, a measuring device 1 according to a preferred embodiment of the present invention comprises a hollow cylindrical sleeve 3 and a cylindrical rod 7. The inner diameter w₂ of the hollow sleeve 3 is slightly larger (e.g. 2.1 mm) than the outer diameter w₃ of rod 7 (e.g. 2.0 mm) such that rod 7 can longitudinally slide within hollow sleeve 3. Both, sleeve 3 and rod 7 are held in holder pieces 21, 23. The holder pieces 21, 23 itself can be held in respective holders 25, 27 by elastic O-rings 29, 31.

The holders 25, 27 to which sleeve 3 and rod 7 are attached, have cams 33, 35 laterally protruding therefrom. When assembled, these cams 33, 35 can be guided within respective grooves 37, 39 formed in a grip 41 serving as rotatable part of handle 11.

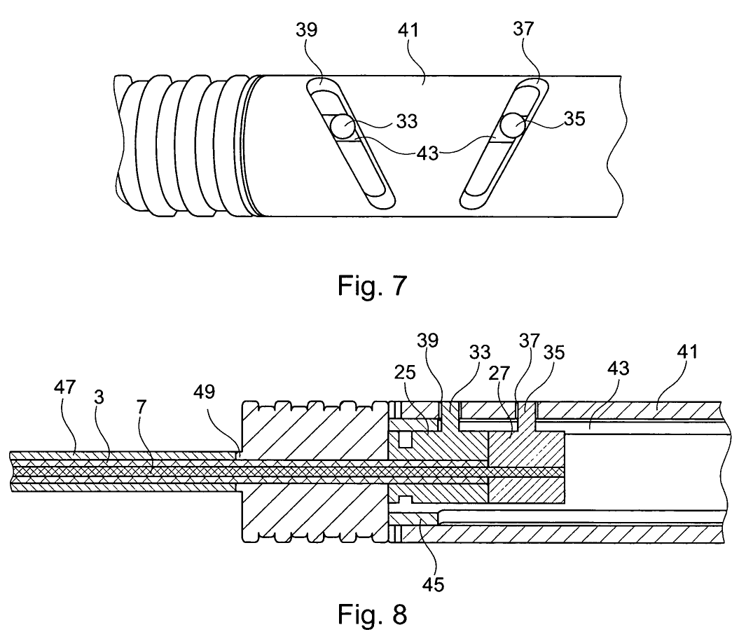

As can also be seen in FIG. 7 showing a side view of the area A indicated in FIG. 5, the two grooves 37, 39 are arranged at an angle with respect to each other. As the cams 33, 35 are guided within grooves 37, 39 and, the cams 33, 35 are guided in a linear groove 43 arranged in a static part 45 of the handle 11, by rotating grip 41 rod or bar 7 can be linearly displaced with respect to sleeve 3 along its longitudinal axis. In that way, the measuring device can be activated by rotating the grip 41 such that rod 7 can either be brought to the distal position where its hook-end 9 protrudes through the opening 5 of the sleeve or to the proximal position, where rod 7 is completely housed within sleeve 3.

On static part 45 of handle 11, a counter-piece 47 can be attached by means of an adapter 49. The adapter 49 as well as holder pieces 21, 23 can be adapted such that counter-pieces 47, rods 7 and/or sleeves 3 of different cross-sections or diameters can be attached to the handle 11. The counter-piece 47 can be a hollow cylinder having an inner diameter w₄ slightly larger than the outer diameter w₁ of sleeve 3. Accordingly, counter-piece 47 can be linearly slit along sleeve 3. Furthermore, on the static part 45, a scale 51 is formed such that the distance between the distal end of the counter-piece 47 and the hook-end 9 of rod 7 can be indicated and easily read through an opening 53 formed in grip 41.

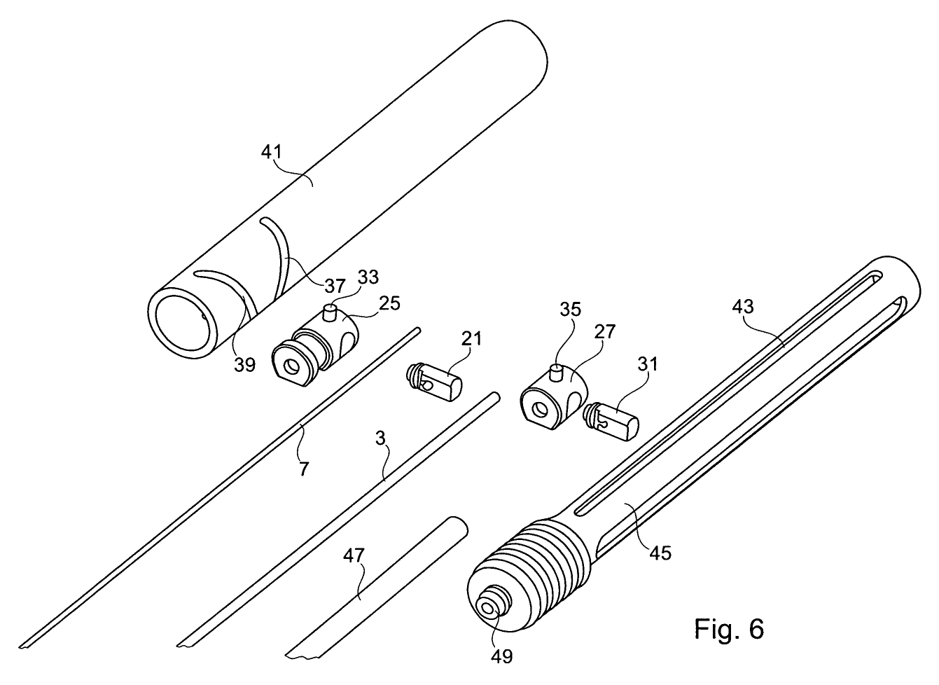

FIG. 8 shows a cross-section of the measuring device from which the interaction of the components shown in FIGS. 5 and 6 can be derived. For clarity purposes, some minor details such as e.g. the holder pieces 21, 23 or features of the adaptor 49 have been omitted in FIG. 8.

For measuring the length of a previously drilled through-hole within a bone of a patient, a surgeon first brings the rod 7 to its proximal position by rotating the grip 41 accordingly. In this withdrawn proximal position, sleeve 3 can be easily inserted through the through-hole. Then, by turning grip 41 accordingly, the surgeon brings rod 7 to its distal position where hook-end 9 protrudes from the lateral surface of sleeve 3. The combination of sleeve 3 and rod 7 is then drawn back until the hook-end laterally abuts the distal surface of the bone. Then, handle 11 including static part 45 with counter-piece 47 attached thereto, can be slid along sleeve 3 until the distal end of the counter-piece 47 abuts to the proximal surface of the bone. The length of the through-hole is then indicated by the number on scale 51 which can be read through the opening 53 in the grip 41.

It should be noted that the term “comprising” does not exclude other elements or steps and the “a” or “one” does not exclude a plurality. Also elements described in association with different embodiments and aspects may be combined. It should also be noted that reference signs in the claims shall not be construed as limiting the scope of the claims.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A measuring device for measuring a length of a through-hole in a bone, the measuring device comprising:
a longitudinally extending elongated hollow sleeve having a bore and a lateral opening adjacent its distal extremity;
a laterally deflectable rod having a hook-end at its distal extremity at least partly housed within the hollow sleeve and is slidable with respect to the sleeve along its longitudinal direction;
a deflection element formed on an inner surface of the sleeve bore opposite the lateral opening in the sleeve such that as the rod is slid to a distal position of the sleeve the rod is deflected radially outwardly and displaced such as to protrude through the opening of the sleeve and beyond an outer surface of the sleeve; and
a rotatable actuator coupled to the hollow sleeve and the rod for sliding the rod with respect to the sleeve.

2. The measuring device according to claim 1, wherein the sleeve is substantially rigid and at least the distal extremity of the rod is substantially flexible such as to be deflectable into a bent configuration to protrude beyond the outer surface of the sleeve.

3. The measuring device according to claim 2, wherein the deflectable element of the hollow sleeve comprises a tilted ramp at its inner side such that the ramp forcing the distal extremity of the bar into the bent configuration when the bar is slid to the proximal position of the sleeve.

4. The measuring device according to claim 1, wherein the sleeve extends longitudinally beyond the position of the hook-end when at its deflected position.

5. The measuring device according to claim 1, wherein a distal end of the sleeve is tapered.

6. The measuring device according to claim 1, further comprising a hollow counter-sleeve slidably receiving the elongated hollow sleeve and for indicating a distance between the hook-end of the rod and a distal end of the counter-sleeve.

7. The measuring device according to claim 1, wherein the actuator comprises a handle attachable to a proximal extremity of the sleeve and of the rod, where the handle is adapted to displace the rod longitudinally along the sleeve upon activation of the handle.

8. The measuring device according to claim 7, wherein the handle includes a rotatable cam portion mounted thereon for engaging a cam element on the rod.

9. The measuring device according to claim 7, wherein the handle comprises an adapter such that the sleeve can be releasably attached to the handle.

10. A bone thickness measuring kit comprising a measuring device according to claim 1 and a plurality of replacement sleeves having different outer cross sections.

11. A measuring device for measuring a length of a through hole in a long bone, the measuring device comprising:
a longitudinally extending tubular sleeve and a lateral opening in a wall of the tubular sleeve and a deflection element formed on a surface of a bore in the sleeve adjacent the opening;
a deflectable rod slidably received within the bore of the tubular sleeve and having an end for engaging the defection element; and
a rotatable actuator having a cam surface thereon for engaging a cam follower on the deflectable rod such that rotation of the actuator in a first direction causes the end of the deflectable rod to engage the deflection element and extend through the lateral opening in the tubular sleeve to engage bone and rotation in a second direction causes the end of the deflectable rod to move out of the lateral opening in the tubular sleeve to a position within the bore thereof.

12. The measuring device according to claim 11, wherein the sleeve is substantially rigid and at least the distal extremity of the rod is substantially flexible such as to be deflectable into a bent configuration to protrude beyond the outer surface of the sleeve.

13. The measuring device according to claim 12, wherein the deflectable element of the hollow sleeve comprises a tilted ramp at its inner side such that the ramp forcing the distal extremity of the bar into the bent configuration when the bar is slid to the proximal position of the sleeve.

14. The measuring device according to claim 11, wherein the sleeve extends longitudinally beyond the position of the hook-end when at its deflected position.

15. The measuring device according to claim 11, wherein a distal end of the sleeve is tapered.

16. The measuring device according to claim 11, further comprising a hollow counter-sleeve slidably receiving the elongated hollow sleeve and for indicating a distance between the hook-end of the rod and a distal end of the counter-sleeve.

17. The measuring device according to claim 11, wherein the device comprises a handle attachable to a proximal extremity of the sleeve and of the rod.

18. The measuring device according to claim 17, wherein the handle includes the rotatable actuator having a cam portion mounted thereon for engaging the cam element on the rod.

19. The measuring device according to claim 17, wherein the handle comprises an adapter such that the sleeve can be releasably attached to the handle.

20. A bone thickness measuring kit comprising a measuring device according to claim 11 and a plurality of replacement sleeves having different outer cross sections.
